(19) United States
(12) Patent Application Publication
(10) Pub. No.: US 2017/0333031 A1
(43) Pub. Date: Nov. 23, 2017

(54) SUTURE HOLDING SLEEVE FOR LAPAROSCOPIC INSTRUMENTS

(71) Applicant: SALEM AFIF AL-HAQAN, SAFAT (KW)

(72) Inventor: SALEM AFIF AL-HAQAN, SAFAT (KW)

(21) Appl. No.: 15/158,573

(22) Filed: May 18, 2016

(51) Int. Cl. A61B 17/04

(52) U.S. Cl. CPC A61B 17/0483; A61B 17/0469

(57) ABSTRACT

The suture holding sleeve for laparoscopic instruments includes a hollow tubular member having a first opening and a second opening, as well as a smooth inner surface and a knurled outer surface. The knurled outer surface may include a plurality of lines positioned lengthwise along the outer surface of the suture holding sleeve, a plurality of rings positioned along the outer surface of the suture holding sleeve, and/or a combination of the plurality of lines positioned lengthwise and the plurality of rings positioned along the outer surface of the suture holding sleeve.

### BACKGROUND OF THE INVENTION

### 1. FIELD OF THE INVENTION

The present invention relates to surgical instruments, and more particularly to a suture holding sleeve for laparoscopic instruments used for holding a suture during a laparoscopic procedure.

### 2. DESCRIPTION OF THE RELATED ART

Laparoscopic surgery is a surgical technique used by doctors that is minimally evasive to the patient and may involve surgical procedures that include the assistance of a video camera and several thin instruments inserted through small incisions, at a location in the body generally away from the point of incision. Laparoscopic instruments that are used for these surgical techniques are generally known in the art. Typically, laparoscopic instruments may be used as suture passers (i.e. needle holder) and/or graspers/dissectors (e.g. Maryland-type). Such devices generally include a pair of jaws to clamp tissue or skin, and a suturing needle to join body tissue, once the procedure is complete. Occasionally, as a result of the nature of the procedure, the medical practitioner while using a clamping device may lose control of a suture within the tissue or organ of the patient's body, forcing the medical practitioner to use the instrument or other instruments to search for the suture in the body. Finding and re-engaging the suture may be difficult for the practitioner, if hidden under bodily fluids, or inflammation in the body.

Thus, a suture holding sleeve that helps the practitioner to engage and hold a suture in place, during the surgical laparoscopic procedure, is needed to solve the aforementioned problem.

### SUMMARY OF THE INVENTION

The suture holding sleeve for laparoscopic instruments includes a hollow tubular member having a first opening and a second opening, as well as a generally smooth inner surface and a knurled or reticulated outer surface. The knurled outer surface may include a plurality of raised lines positioned lengthwise along the outer surface of the suture holding sleeve, a plurality of raised rings positioned along the outer surface of the suture holding sleeve, or a combination of a plurality of raised lines positioned lengthwise and a plurality of raised rings positioned along the outer surface of the suture holding sleeve.

These and other features of the present invention will become readily apparent upon further review of the following specification and drawings.

### BRIEF DESCRIPTION OF THE DRAWINGS

FIG. 1 is an environmental, perspective view of an embodiment of a suture holding sleeve, connected to a laparoscopic instrument, according to the present invention.

FIG. 2 depicts the holding sleeve for a laparoscopic instrument illustrated in FIG. 1, according to the present invention.

FIG. 3 is another embodiment of a holding sleeve for a laparoscopic instrument, according to the present invention.

FIG. 4 is another embodiment of a holding sleeve for a laparoscopic instrument, according to the present invention.

Similar reference characters denote corresponding features consistently throughout the attached drawings.

### DETAILED DESCRIPTION OF THE PREFERRED EMBODIMENTS

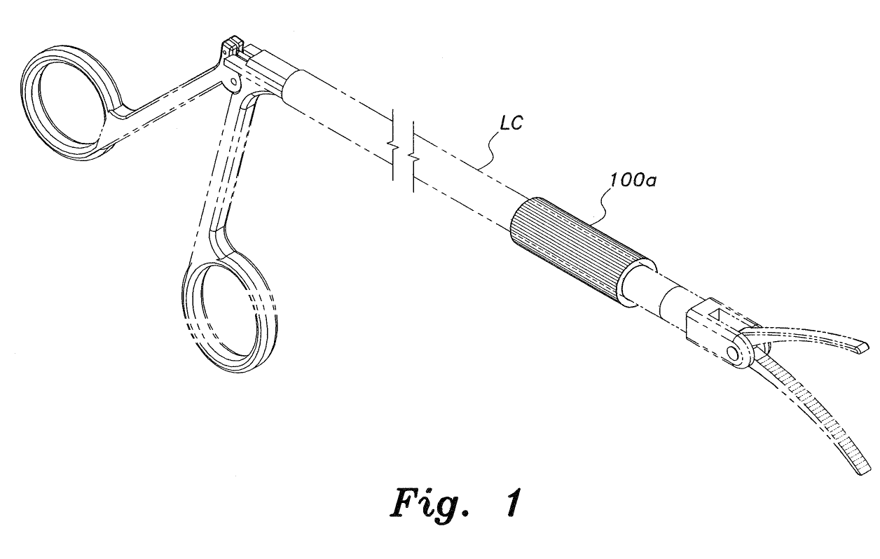

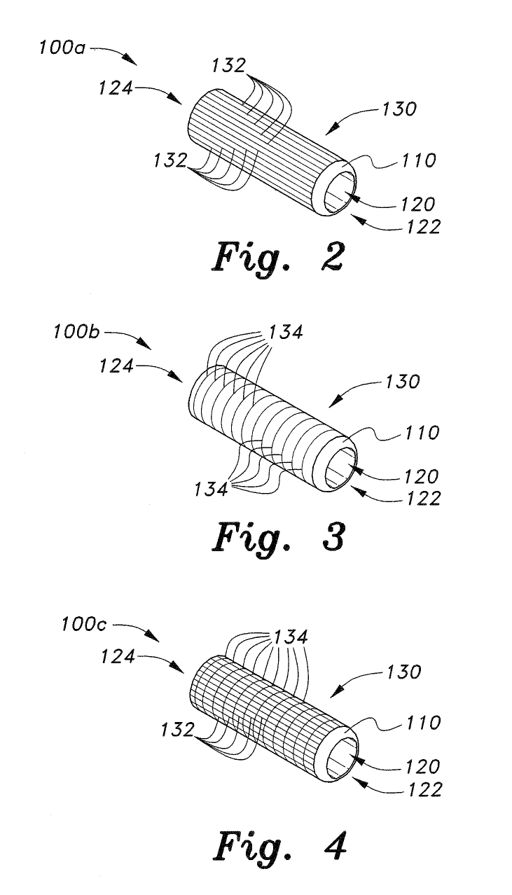

FIGS. 1-4 illustrate several embodiments of a suture holding sleeve for a laparoscopic instrument, in accordance with the claimed invention. Referring now to FIGS. 1 and 2, there an embodiment of a suture holding sleeve, generally designated at 100a, is shown. FIG. 3 illustrates another embodiment of the suture holding sleeve, generally designated at 100b. FIG. 4 illustrates another embodiment of a suture holding sleeve, generally designated at 100c.

Each of the illustrated suture holding sleeve embodiments 100a, 100b, 100c generally include a hollow tubular member 110 having a first opening 122 provided at one end of the suture holding sleeve 100a, 100b, 100c, and a second opening 124 provided at an opposing end of the suture holding sleeve 100a, 100b, 100c. The sleeve 100a, 100b, 100c further includes a generally smooth inner surface 120, and a generally knurled outer surface 130, configured to enhance a user's grip on the laparoscopic instrument and a suture. As illustrated in FIG. 1, the smooth inner surface 120 permits the suture holding sleeve 100a, 100b, 100c to be slid onto a laparoscopic instrument, such as a laparoscopic clamp LC, for example. As depicted in FIGS. 1 and 2, the knurled outer surface 130 may include a plurality of raised surfaces or lines 132 having a linear configuration 132, and positioned lengthwise about the outer surface 130 of the suture holding sleeve 100a. As depicted in FIG. 3, the knurled outer surface 130 may include a plurality of lateral raised surfaces or rings 134 positioned along the outer surface 130 of the suture holding sleeve 100b. As illustrated in FIG. 4, the knurled outer surface 130 may include a combination of a plurality of raised lines 132 positioned lengthwise and a plurality of raised rings 134, positioned along the outer surface 130 of the suture holding sleeve 100c.

The suture holding sleeve 100a, 100b, 100c may be comprised of a silicone rubber or similar type of elastic rubber material, formed from a suitable type of polymer. As such, the sleeve 100a, 100b, 100b provides sticky or adhesive properties, conducive for the suture holding sleeve 100a, 100b, 100c to fit onto a variety of of laparoscopic instruments, as well as adhere to the suture needle and/or suture within the patient's tissue or organ. The suture holding sleeve 100a, 100b, 100c may have any length, however, it is contemplated that the sleeve may have a length of approximately 10 cm. As such, the sleeve 100a, 100b, 100c length is suitable to create surface area large enough to grasp or adhere to a suture without difficulty. It is also contemplated that the suture holding sleeve 100a, 100b, 100c may have any diameter. However, it is further contemplated that the sleeve 100a, 100b, 100c may have a diameter ranging between 5 cm to 10 cm, suitable to fit onto a selected laparoscopic instrument, such as the laparoscopic clamp LC, illustrated in FIG. 1. It is further contemplated that the diameter of the suture holding sleeve 100a, 100b, 100c may vary to accommodate laparoscopic instruments having a variety of thicknesses. The thickness of the suture holding sleeve 100a, 100b, 100c may range between 1 cm to 2 cm.

In operation, prior to commencing laparoscopic surgery, a medical practitioner places the suture holding sleeve 100a, 100b, 100c onto the laparoscopic instrument, such as a laparoscopic clamp LC, as illustrated in FIG. 1. It is to be noted that although the suture holding sleeve 100a, 100b, 100c is typically positioned near the tip of the laparoscopic instrument, it is contemplated that the suture holding sleeve 100a, 100b, 100c may be positioned virtually anywhere along the laparoscopic instrument in order to aid the medical practitioner during the laparoscopic suturing process. On occasion, as a result of the tight spaces created and fluids secreted during laparoscopic surgery, the medical practitioner may find it difficult to suture an incision. As such, the suture holding sleeve 100a, 100b, 100c may be used to assist the practitioner in maintaining control of the laparoscopic instrument.

After the laparoscopic surgery has been completed, the medical practitioner typically uses sutures to close the tissue or the incision made for the surgery. If, for some reason, the medical practitioner loses control of the suture while closing up the tissue or the incision, he or she may use the suture holding sleeve 100a, 100b, 100c to engage and adhere to the suture. This will be helpful in circumstances in which the practitioner has problems seeing the suture and/or grasping suture with the alligator clips of the laparoscopic instrument. Once the practitioner engages the suture with the knurled outer surface 130 of the suture holding sleeve 100a, 100b, 100c and has control of the suture, the medical practitioner may remove the sleeve 100a, 100b, 100c from the tissue or incision and, subsequently, grasp the suture with the instrument LC, to complete the closure of the tissue and/or the incision.

It is to be understood that the present invention is not limited to the embodiments described above, but encompasses any and all embodiments within the scope of the following claims.

1. in combination, a laparoscopic clamp instrument and a suture holding sleeve for the laparoscopic clamp instrument, the combination consisting of:
the laparoscopic clamp instrument, wherein the laparascopic clamp has an elongated body including a tissue-retracting clamp mechanism at one end and a handle at the other end to manipulate the tissue-retracting clamp mechanism; and
the elongated elastic hollow tubular member having a thickness of between 1 cm to 2 cm, the tubular member having a first opening and a second opening, the hollow tubular member having a continuous smooth inner surface configured to receive the laparoscopic instrument therein, and a continuous knurled outer surface configured to engage a suture, wherein the continuous smooth inner surface and the continuous knurled outer surface extend from the first opening to the second opening.

2. The combination according to claim 1, wherein the knurled outer surface comprises a plurality of spaced parallel lines positioned lengthwise about the outer surface of the suture holding sleeve.

3. The combination according to according to claim 1, wherein the knurled outer surface comprises a plurality of rings positioned transversely along the outer surface of the suture holding sleeve.

4. The combination according to according to claim 1, wherein the knurled outer surface comprises a combination of: a plurality of spaced parallel lines positioned lengthwise about the outer surface of the suture holding sleeve and a plurality of rings positioned transversely along the outer surface of the suture holding sleeve.

5. The combination according to according to claim 1, wherein the suture holding sleeve is formed from silicone.
